(19) United States
(12) Patent Application Publication
(10) Pub. No.: US 2025/0236577 A1
(43) Pub. Date: Jul. 24, 2025

(54) METHANOL DISTILLATION SYSTEM WITH ENHANCEMENTS FOR IMPROVED EFFICIENCY

(71) Applicant: CRI, HF, Kopavogur (IS)

(72) Inventors: Armen KODJAIAN, Buenos Aires (AR); Francesco PAGLIARO, Oggiono (IT); Petur Mar GISLASON, Kopavogur (IS); Olafur Hrafn BJORNSSON, Reykjavik (IS)

(21) Appl. No.: 18/986,261

(22) Filed: Dec. 18, 2024

Related U.S. Application Data
(60) Provisional application No. 63/624,046, filed on Jan. 23, 2024.

(51) Int. Cl. C07C 29/80; C07C 29/78

(52) U.S. Cl. CPC C07C 29/80; C07C 29/78

(57) ABSTRACT

Disclosed herein are systems and embodiments for improving energy efficiency of a chemical separations plant through heat integration. Heat integration may provide heating loops between distillation columns and/or integrated electric heating components configured to supply heat to reboilers in the chemical separations plant. Heat integration may use heat from an associated chemical synthesis plant configured to carry out an exothermic reaction (e.g., production of methanol from CO₂ and H₂). Such integrated process designs allow for the use of “low-grade” heat, thereby improving energy efficiency. Furthermore, such integrated process designs allow for electrification of exemplary chemical separations plants, replacing the use of steam as a heating medium with more energy efficient and carbon-emission neutral, electrical methods.

### CROSS REFERENCE TO RELATED APPLICATION(S)

The present application claims the benefit of U.S. Provisional Patent Application Ser. No. 63/624,046 filed on Jan. 23, 2024 entitled “METHANOL DISTILLATION SYSTEM WITH ENHANCEMENTS FOR IMPROVED EFFICIENCY”, which is herein incorporated by reference in its entirety.

### FIELD OF THE DISCLOSURE

The disclosure relates to new process designs and systems of heat integration designed to improve energy efficiency of such plants. By way of example, such integration may be between (i) a chemical synthesis plant that produces heat from an exothermic reaction (e.g., from methanol synthesis) in connection with (ii) a chemical purification plant that requires energy to isolate a value-added product such as high purity distillate methanol. The system may exchange heat between such plants, to better maximize efficiency, reduce waste heat, reduce cooling load, etc. Furthermore, the process designs and systems of heat integration described herein can fully or partially displace the need for steam as a heat source.

### BACKGROUND

Global climate change has been deemed to be the “most pressing environmental challenge of our time.” The National Aeronautics and Space Administration (NASA) cites that “scientific evidence for warming of the climate system is unequivocal.” Climate change results from the warming effects of greenhouse gases such as water vapor, nitrous oxide, methane, and carbon dioxide. Of these, carbon dioxide emissions are a key culprit, as global atmospheric concentration of CO₂ has increased by a third since the Industrial Revolution began. CO₂ emissions largely stem from human activities, such as the combustion of fossil fuels, the byproducts of which are emitted into the atmosphere.

Human industry, across the board, has been under sustained pressure to reduce their carbon footprint. Proposed methods include carbon capture technologies as well as a shift towards renewable sources of energy that do not emit CO₂ as a byproduct of their use. Converting CO₂ into methanol represents a promising method of carbon capture that can bring down net emissions and store renewable energy in chemical bonds. Methanol, which is a vital precursor for transportation fuels, industrial chemicals such as formaldehyde, as well as plastics, paints, textiles, and other compositions, is an effective alternative for reusing CO₂, or reducing CO₂ emissions Methods for storing renewable energy in methanol's chemical bonds solves numerous transportation-related problems of renewable electricity. For instance, the favorable energy density and liquid state of methanol, its compatibility with transportation fuels, and lower losses during transportation render methanol an advantaged energy-storage medium.

Further, given the massive global scale of methanol demand—200,000 tons per day and growing—diverting CO₂ from the atmosphere and into methanol has the potential to recycle a large quantity of the greenhouse gases responsible for climate change. Methanol also has significant potential for growth as a transportation fuel. Another increasing disposition for methanol includes methanol-to-olefins processes that convert methanol into the building blocks of polyolefins, the most common plastic product for which there is an ever-growing global demand.

Methanol is typically produced in industrial settings from synthesis gas (“syngas”), a combination of varying amounts of H₂, CO, and CO₂ frequently derived from gasified coal. Methanol synthesis is an exothermic reaction, causing methanol synthesis plants to produce large amounts of thermal energy. Methanol produced in such a manner typically contains unwanted contaminates such as water and dissolved gases like CO and CO₂, herein described as crude methanol. As a result, crude methanol needs to be purified.

Purifying methanol is an energy intensive process that requires heat input to separate methanol from water and other byproducts. Methanol purification is typically performed in a distillation column and associated equipment, demanding approximately 4.2 MJ/kg of purified methanol produced. This high energy input cuts into potential reductions in a plant's carbon footprint, signaling a need for methods and/or systems that use energy more efficiently, to reduce any required net energy input.

### BRIEF SUMMARY

The present disclosure is directed to systems and methods that provide for improved use of heat energy between processes by integrating heat between them. For example, in one specific implementation, excess heat from an ETL plant (e.g., Applicant's emissions to liquid (ETL) plant) that converts CO₂ emissions and hydrogen (e.g., from water electrolysis) to produce methanol can be integrated with a methanol purification plant. While described in such a context, it will be appreciated that integration may be possible in a variety of other chemical synthesis or other plants. By way of example, heat from the ETL plant, which is generated due to methanol synthesis being an exothermic reaction, can be utilized in the distillation process of the methanol purification plant, e.g., to power the reboilers of a distillation column, e.g., heating a methanol/water/contaminates mixture to power the distillation process.

Heat from heat generating plants is often characterized based on temperature. Higher temperature heat is said to be of higher quality or “grade”. In the present disclosure, “low-grade” heat is considered to be heat that is less than about 150° C. and “high-grade” heat is considered to be heat that is greater than about 150° C. 150° C. is a typical temperature required to power the methanol purification process that may be used with embodiments described herein. Low-grade heat is often considered non-economical to utilize and is generally dissipated to the environment during many industrial processes, as “waste heat”. The present disclosure describes new methods and processes for designing chemical separations plants that can economically make use of low-grade heat as a heat source, within certain portions of the process. Specifically, the embodiments described herein open or identify low temperature windows in a chemical separations plant that allow for the use of low-grade heat to partially or fully power energy intensive steps through heat integration with another portion of the overall process that produces low-grade heat. Furthermore, the embodiments described herein may include internal heat integration between different components of a chemical separations plant, allowing for improved energy efficiency and negating the need for supplemental, external energy input.

In an embodiment, a chemical separations plant is designed to open up low-temperature windows that can utilize low-grade heat as a heat source through a three-column separations design, wherein the chemical separations plant comprises a first column, a second column and a third column. The first column is configured to receive a chemical process stream (e.g., a crude methanol stream), the first column further comprising at least one condensing unit, e.g., located at an overhead portion of the column, and at least one reboiler configured to receive low-grade heat (e.g., low-grade heat from a ETL or other exothermic chemical synthesis plant). The second column is configured to receive a bottoms fraction from the first column, the second column further comprising at least one condensing unit, e.g., located at an overhead portion of the column, at least one reboiler configured to receive heat from at least one condensing unit of the third column. The third column is configured to receive a bottoms fraction from the second column, the third column further comprising at least one condensing unit, e.g., located at an overhead portion of the column, and at least one reboiler configured to receive high-grade heat (e.g., high-grade heat from an ETL or other exothermic chemical synthesis plant, or otherwise).

This embodiment distributes the duty required to separate a chemical process stream across three columns, opening low temperature windows in the reboilers of columns that receive a chemical process stream with a higher concentration of volatile materials. In other words, within the first column, where higher concentration volatile materials are present, lower grade heat can be employed to produce some fraction of the purified product stream (e.g., purified methanol), rather than higher grade heat that will be needed within the downstream separations columns.

In another exemplary embodiment, a chemical separations plant is designed to open up low-temperature windows that can utilize low-grade heat as a heat source through a side-reboiler design, wherein the chemical separations plant comprises: a first column configured to receive a chemical process stream (e.g., a crude methanol stream), wherein the first column further comprises at least one condensing unit, e.g., located at an overhead portion of the column, and at least one reboiler configured to receive heat from at least one condensing unit of the second column; a second column configured to receive a bottoms fraction from the first column, wherein the second column further comprises at least one condensing unit, e.g., located at an overhead portion of the column, a first reboiler associated with a bottom portion of the second column, and a second reboiler associated with a middle portion of the second column; a plurality of heat exchangers, wherein a first heat exchanger is configured to deliver a source of high-grade heat to the first reboiler of the second column (e.g., high-grade heat from an ETL or other exothermic chemical synthesis plant, or otherwise), a second heat exchanger that is configured to deliver a source of low-grade heat (e.g., low-grade heat from a ETL or other exothermic chemical synthesis plant) to the second reboiler of the second column, and a third heat exchanger that is configured to deliver heat from the condensing unit of the second column to the reboiler of the first column.

This embodiment opens a low-temperature window in the second reboiler of the second column by providing the second reboiler at the middle portion of the second column (i.e., the second reboiler introduces the heated stream into the middle portion of the second column, rather than at the bottom portion of the column). This design adds an additional reboiler that delivers a stream that is cooler (e.g., at around 90° C. to 140° C., around 100° C. to 130° C. or around 110° C. to 120° C.) than the stream delivered by the first reboiler (e.g., at around 125° C. to 175° C., around 135° C. to 165° C. or around 145° C. to 155° C.). Due to the placement and reduced temperature of the fraction being provided to the second column by the second reboiler (the side reboiler), a low-grade heat source can be used to contribute part of the heat needed in the distillation process, rather than requiring high-grade heat for the entire feed stream that would otherwise be fed into the column through the first reboiler.

These exemplary embodiments can be integrated with a portion of the plant that can act as a high-grade and low-grade heat source. In an embodiment, a reactor configured to carry out an exothermic reaction and produce high-grade heat (e.g., at around 210° C. to 310° C., around 230° C. to 290° C. or around 250° C. to 270° C.) is coupled to a first heat exchanger. This first heat exchanger is coupled to a reboiler in an integrated chemical separations plant that requires high-grade heat (e.g., typically around 170° C. to 180° C.). By way of example, such a reboiler needing high-grade heat could be the reboiler of the third column in the described three-column plant, or the first reboiler of the second column in the described side-reboiler plant). In any case, such a configuration can fill the need of supplying high-grade heat to the integrated separations plant. A second heat exchanger is provided downstream of the first heat exchanger, where the second heat exchanger may handle the same material stream as the first heat exchanger, but wherein the high-grade heat has been extracted from the process stream. The second heat exchanger is coupled to a low-temperature window in an integrated chemical separations plant (e.g., the reboiler of the first column in the described three-column plant or the second reboiler of the second column in the described side-reboiler plant), supplying low-grade heat (e.g., at a temperature of about 120° C. to about 135° C.) within the integrated chemical separations plant, where such low-grade heat can be employed, to provide at least some of the needed heat. The configuration identifies a low temperature window where such heat can be provided, and uses the low-grade heat to meet such need. By integrating a chemical synthesis plant with a chemical separations plant that can utilize low-grade heat, the integrated plant can experience a significant duty decrease, e.g., from about 4.2 MJ/kg of produced high purity methanol, to zero or near zero (e.g., less than 2, less than 1, less than 0.5, or less than 0.1 MJ/kg of produced high purity methanol).

In some embodiments, heat from a partnered chemical synthesis plant can be delivered to the reboilers of a chemical separations plant through a direct-process heating system, wherein hot gases or other fluids produced by a reactor can be coupled directly to the reboilers of a chemical separations plant.

In some embodiments heat from a partnered chemical synthesis plant can be delivered to the reboilers of a chemical separations plant through a system of one or more heating medium loops, wherein hot gases or other fluids produced by a reactor can be coupled to such a heating medium loop, wherein the heating medium loops are further coupled to the reboilers of a chemical separations plant, providing heat thereto.

Furthermore, some chemical separations plants seek to reduce their carbon footprint by using renewable energy instead of burning fossil fuels for needed heat or power. Additionally, access to readily available steam as a heat source in some facilities can be challenging. The embodiments described herein can reduce or eliminate the need to use steam as a heat source for chemical separations plants and assist in the electrification of chemical separations plants. Described below are additional embodiments that electrify exemplary chemical separations plants, reducing or negating the need to use steam as a heat source.

In an exemplary embodiment, a chemical separations plant is designed to use electricity as a source of power and reduce or eliminate steam as a heat source, the chemical separations plant comprising: a first column configured to receive a chemical process stream, the first column further comprising at least one condensing unit, e.g., located at an overhead portion of the column and at least one reboiler; a second column configured to receive a bottoms fraction from the first column, the second column further comprising at least one condensing unit located at the overhead portion of the column and at least one reboiler; a heat exchanger configured to transfer heat to the reboiler of the first column from the condensing unit of the second column; and a heat source configured to provide heat to the reboiler of the second column.

In some embodiments, the heat source can be a hot oil loop.

In some embodiments, the heat source includes an electric reboiler, wherein the reboiler of the second column further comprises electric coils configured to supply heat to the reboiler.

In some embodiments, the heat source includes an electrified heating medium loop comprising tubing coupled to the reboiler of the second column, a pump, and an electric heater.

In some embodiments, the heat source includes a heat pump, comprising tubing coupled to the reboiler of the second column, a compressor, and an expansion valve.

It will be appreciated that the embodiments described herein are not limited to methanol purification processes, but may be used in various other chemical processes where improved systems and/or methods for the separation of products from a process stream is required or desired. Specific methods, embodiments, and variations of the system are described in greater detail in the following detailed description. Furthermore the heat producing portion of such plants is not limited to embodiments where the heat producing portion of the plant is a methanol synthesis process. A wide variety of chemical synthesis processes (e.g., that are exothermic), e.g., due to an exothermic chemical reaction could be used.

By way of further example, an exemplary chemical separations plant may include a first column, a second column, and a third column, wherein the first column is configured to receive a chemical process stream, the first column further comprising at least one associated condensing unit, (e.g., located at an overhead portion of the first column), and at least one reboiler configured to receive low-grade heat from a low-grade heat source. The second column may be configured to receive at least a portion of a bottoms condensate fraction from the first column, the second column further comprising at least one associated condensing unit, (e.g., located at an overhead portion of the second column), and at least one reboiler configured to receive heat from a top fraction of the third column. The third column may be configured to receive at least a portion of a bottoms condensate fraction from the second column, the third column further comprising at least one associated condensing unit, (e.g., located at an overhead portion of the third column), and at least one reboiler configured to receive high-grade heat from a high-grade heat source.

In any of the described embodiments, the first column may further comprise at least one stripper unit located at the overhead portion of the first column.

In any of the described embodiments, the chemical process stream may comprise crude methanol.

In any of the described embodiments, the first column may produce from about 25% to about 40% of purified methanol produced by the chemical separations plant.

In any of the described embodiments, the second column may produce from about 30% to about 50% of purified methanol produced by the chemical separations plant.

In any of the described embodiments, the third column may produce from about 20% to about 30% of purified methanol produced by the chemical separations plant.

In any of the described embodiments, the first column may be a low-pressure column.

In any of the described embodiments, the second column may be a low-pressure column.

In any of the described embodiments, the third column may be a medium pressure column (e.g., it may operate at a pressure that is higher than the operating pressure of the first and/or second columns).

In any of the described embodiments, the high-grade heat source may be a stream of hot gas or other hot fluid produced by a chemical synthesis plant.

In any of the described embodiments, the low-grade heat source may be a stream of hot gas or other hot fluid produced by a chemical synthesis plant, wherein any high-grade heat initially present in the stream of hot gas or other hot fluid has been at least partially depleted.

In any of the described embodiments, a heating medium loop may transfer high-grade heat from the high-grade heat source to the at least one reboiler of the third column.

In any of the described embodiments, a heating medium loop may transfer low-grade heat from the low-grade heat source to the at least one reboiler of the first column.

In any of the described embodiments, the low-grade heat source from which heat is recovered may be a hot wastewater stream.

Another exemplary embodiment is directed to a system for heat integration of (i) a chemical synthesis plant which generates low-grade heat and high-grade heat from an exothermic reaction and (ii) any of the chemical separations plants described herein. Such a system may include a reactor for carrying out the chemical synthesis configured to produce a process stream, a first heat exchanger configured to transfer high-grade heat from the process stream to the reboiler of the third column, and a second heat exchanger positioned downstream from the first heat exchanger and configured to transfer remaining low-grade heat from the process stream to the reboiler of the first column.

Another exemplary embodiment is directed to a chemical separations plant including a first column configured to receive a chemical process stream, wherein the first column further comprises at least one condensing unit, (e.g., located at an overhead portion of the first column), and at least one reboiler. A second column is also provided, configured to receive at least a portion of a bottoms condensate fraction from the first column, wherein the second column further comprises at least one condensing unit, (e.g., located at an overhead portion of the second column), a first reboiler associated with a bottoms fraction of the second column, and a second reboiler associated with a middle fraction of the second column. A plurality of heat exchangers are also provided, wherein a first heat exchanger is configured to deliver a source of high-grade heat to the first reboiler of the second column, a second heat exchanger is configured to deliver a source of low-grade heat to the second reboiler of the second column, and a third heat exchanger is configured to deliver heat from a top fraction produced from the second column to the at least one reboiler of the first column.

In any of the described embodiments, the first column may further comprise at least one stripper unit located at the overhead portion of the first column.

In any of the described embodiments, the chemical process stream may comprise crude methanol.

In any of the described embodiments, the first column may be a low-pressure column.

In any of the described embodiments, the second column may be a medium pressure column (e.g., operating at a higher pressure than the first column).

Another exemplary embodiment may be directed to a system for heat integration of (i) a chemical synthesis plant which generates low and high-grade heat from an exothermic reaction and (ii) any chemical separations plant as described herein. Such a system may include a reactor for carrying out the chemical synthesis configured to produce a process stream, a first heat exchanger configured to transfer high-grade heat from the process stream to the first reboiler of the second column, and a second heat exchanger configured downstream from the first heat exchanger and configured to transfer remaining low-grade heat from the process stream to the second reboiler of the second column.

Another exemplary embodiment may be directed to a chemical separations plant including a first column configured to receive a chemical process stream, the first column further comprising, e.g., at least one condensing unit, (e.g., located at an overhead portion of the first column), and at least one reboiler. A second column may be provided, configured to receive at least a portion of a bottoms condensate fraction from the first column, the second column further comprising at least one condensing unit, (e.g., located at an overhead portion of the second column), and at least one reboiler. A heat exchanger may be provided, configured to transfer heat to the at least one reboiler of the first column from a top fraction produced from the second column, and an electric heat source may be provided, configured to provide heat to the at least one reboiler of the second column.

In any of the described embodiments, the electric heat source may comprise a hot oil loop comprising tubing coupled to the at least one reboiler of the second column, a pump, and an electric heater.

In any of the described embodiments, the electric heat source may comprise an additional reboiler that further comprises an internal electric heater.

In any of the described embodiments, the electric heat source may comprise a heating medium loop comprising tubing coupled to the at least one reboiler of the second column, a pump, and an electric heater.

In any of the described embodiments, the electric heat source may comprise a heat pump comprising tubing coupled to the at least one reboiler of the second column, a compressor, and an expansion valve.

In any of the described embodiments, the electric heat source may not comprise or use steam.

In any of the described embodiments, the chemical separations plant may be a methanol distillation plant.

In any of the described embodiments, the first column may further comprise at least one stripper unit located at an overhead portion of the first column.

These and other features, aspects, and advantages of the present disclosure will become readily apparent and better understood in view of the following description, appended claims, and accompanying drawings.

### BRIEF DESCRIPTION OF THE DRAWINGS

FIG. 1A schematically illustrates an exemplary chemical separations plant designed to utilize low-grade heat using a three-column design and direct process heating.

FIG. 1B schematically illustrates a chemical separations plant similar to that of FIG. 1A, but that includes a heating medium loop instead of direct process heating.

FIG. 2 schematically illustrates an exemplary chemical separations plant designed to utilize low-grade heat through implementation of a side-reboiler design.

FIG. 3A schematically illustrates a system that integrates the chemical separations plant of FIG. 1A with a methanol synthesis (e.g., ETL) plant.

FIG. 3B schematically illustrates a system that integrates the chemical separations plant of FIG. 1B with a methanol synthesis (e.g., ETL) plant.

FIGS. 3C.1, 3C.2 and 3C.3 schematically illustrate how the direct transfer of heat from a methanol loop or other chemical synthesis to a purification portion of the system is achieved.

FIG. 4A schematically illustrates a system that integrates the chemical separations plant of FIG. 2 with a methanol synthesis (e.g., ETL) plant.

FIGS. 4B and 4C schematically illustrate systems similar to FIGS. 2 and 4A respectively, but in which a heat pump is incorporated to upgrade low-grade heat, without otherwise changing the heating medium loop.

FIG. 5 schematically illustrates a chemical separations plant that utilizes steam to power a chemical separations process.

FIG. 6 schematically illustrates a chemical separations plant that utilizes a hot oil loop to provide heat to the chemical separations process.

FIG. 7 schematically illustrates a chemical separations plant that utilizes an electric reboiler to provide heat to the chemical separations process.

FIG. 8 schematically illustrates a chemical separations plant that utilizes an electrified heating medium loop to provide heat to the chemical separations process.

FIGS. 9A-9B schematically illustrate chemical separations plants that utilize a heat pump to provide heat to the chemical separations process.

### DETAILED DESCRIPTION OF VARIOUS EMBODIMENTS

A better understanding of different embodiments of the disclosure may be had from the following description read with the accompanying drawings in which like reference characters refer to like elements.

While the disclosure is susceptible to various modifications and alternative constructions, certain illustrative embodiments are shown in the drawings and will be described below. It should be understood, however, there is no intention to limit the disclosure to the embodiments disclosed, but on the contrary, the intention is to cover all modifications, alternative constructions, combinations, and equivalents falling within the spirit and scope of the disclosure and defined by the appended claims.

It will be understood that, unless a term is defined in this patent to possess a described meaning, there is no intent to limit the meaning of such term, either expressly or indirectly, beyond its plain or ordinary meaning.

All heat exchangers described herein may be of any suitable heat exchanger configuration having any suitable properties and operated in any suitable manner. By way of non-limiting example, any given heat exchanger may be a parallel-flow heat exchanger, counter-flow heat exchanger, finned or un-finned tubular heat exchanger, shell-and-tube heat exchanger, U-tube heat exchanger, single-pass straight heat exchanger, two-pass straight heat exchanger, plate or frame heat exchanger, plate-fin heat exchanger, microchannel heat exchanger, or otherwise.

Heat integration as a general term will be used to mean any process by which heat from one process or system is introduced into another process or system. The heat integration described in the present embodiments may be internal, meaning within a single independent system or process, or external, meaning that the heat from one independent system or process is introduced into another independent process or system. By independent, it is meant that the system or process can be (and typically would be) conducted independently from, and without regard to or integration with the other system or process (e.g., no exchange of material streams required between such processes).

Although an emissions to liquid (ETL) plant which converts carbon dioxide and hydrogen (e.g., from water electrolysis) into methanol is principally described in the present embodiments as a plant that can benefit from the described configurations, it will be appreciated that other plants characterized by an exothermic chemical reaction (so as to have available waste heat) may also be suitable candidates. For example, other types of chemical synthesis plants that conduct exothermic reactions as part of product synthesis, particularly those that employ a flue gas CO₂ stream as a reactant material used in product synthesis may be suitable candidates for heat integration as described herein.

Likewise, a chemical distillation plant which separates high purity distillate methanol (e.g., about 99% pure) from a crude methanol process stream is principally described in the present embodiments as a plant that can benefit from the described configurations. That said, it will be understood that other chemicals, including other alcohols or other chemical materials that may require purification and/or separation may also benefit from the described configurations.

Low-grade heat as used herein refers to heat that may typically be considered waste heat, or heat with limited or no utility for a given process. High-grade heat as used herein refers to heat sources at higher temperatures, recognized as useful for heating a given stream, within a given process. By way of example, low-grade heat may refer to a heat source that is at less than about 150° C., while high-grade heat may refer to a heat source that is at greater than about 150° C.

The use of the term low temperature window as used herein refers to a step in a given overall process (e.g., purification of methanol) that is identified as capable of utilizing a low-grade heat source to provide needed heat. Such low temperature windows are characterized by a lower temperature, demanding lower quality of energy as opposed to steps of the process that require a high temperature (higher quality of energy) input.

The process of integrating available heat sources and needs for heat is beneficial, especially where one can identify or open low temperature windows, where low-grade heat can be used, as it increases the overall efficiency of energy use, e.g., decreasing the need for external heat in the chemical separations portion of the process, while also, decreasing the need for cooling water in the ETL or other chemical synthesis portion of the process. These benefits can be realized simultaneously, as both high-grade heat and low-grade heat from the ETL plant or other chemical synthesis plant can used in the reboilers of a chemical separations plant, providing the necessary heat to achieve the desired purification or separations process, while reducing the need for externally supplied heat. For example, methanol purification is a costly process that typically demands approximately 4.2 MJ/kg of produced methanol. The presently described heat integration processes allow for a significant duty decrease, e.g., potentially substantially eliminating the typical demand of about 4.2 MJ/kg of produced high purity methanol, through the described heat integration configurations.

Furthermore, by utilizing high-grade heat from a methanol synthesis reactor (or other similar exothermic reactor) and low-grade heat that is otherwise rejected to the atmosphere or other cooling system, the present embodiments may enable a chemical separations plant to run entirely on the heat produced by the associated chemical synthesis plant (e.g., a methanol synthesis plant). Such embodiments can displace the need for steam, typically used to power chemical separations plants. The present embodiments can therefore be useful for plants that may not have access to readily available steam, or are seeking to reduce their carbon footprint, as steam generation often requires a heavy fossil fuel input.

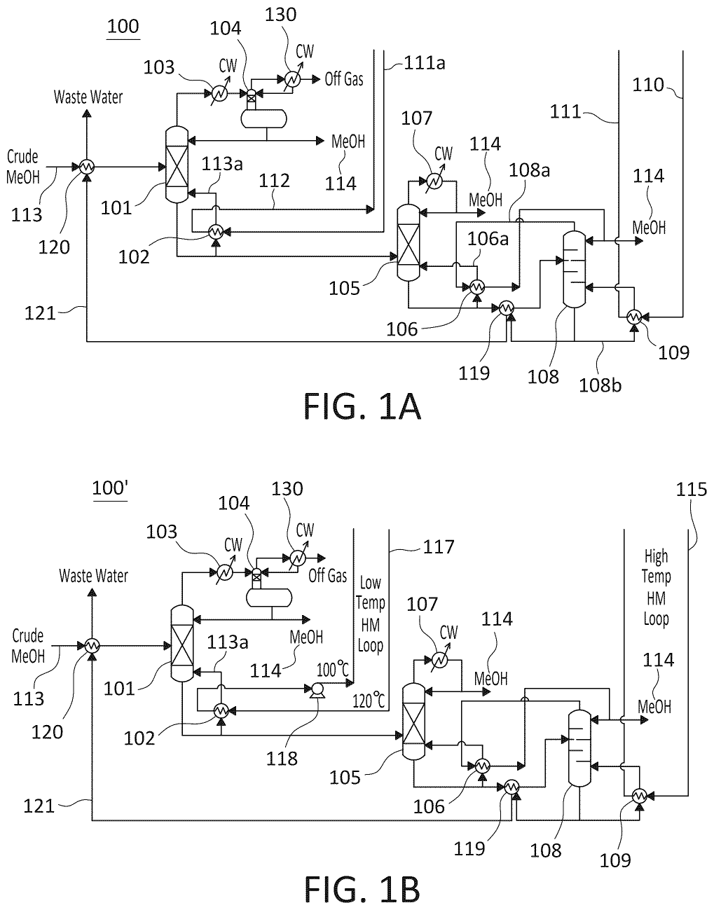

FIG. 1A illustrates an exemplary operational scheme for an exemplary chemical separations plant 100 separating a process stream 113 (e.g., a process stream comprising crude methanol) through a first column 101, a second column 105, and a third column 108. Column 101 may be configured to receive process stream 113 through a side inlet. Column 101 further comprises a reboiler 102 (e.g., associated with a bottoms fraction of column 101) and a condensing unit 103 (associated with an overhead portion of column 101). At least a portion of bottom portion from column 101 is fed to reboiler 102, which is configured to receive low-grade heat (e.g., from a heat source at about 120° C. to about 135° C.), that is external to the separations process (e.g., low-grade heat from an integrated ETL or other chemical synthesis plant) through stream 111a. The low-grade heat is sufficient to reboil the recycled bottoms stream. Heated process stream 113a is returned to column 101, forming a vaporous fraction and a bottoms condensate fraction inside column 101. The vaporous fraction is collected by condensing unit 103 and the bottoms condensate fraction settles to the bottom of first column 101, where a portion of such stream is fed to column 105 and another portion is recycled through reboiler 102. The condensing unit 103 can be coupled to a Stripper unit 104 that functions to remove dissolved gases from the condensed vaporous fraction, producing high purity distillate methanol 114 which is sent to storage or other desired use. An example of an acceptable Stripper unit for use in the embodiments described herein is described in Applicant's U.S. Pat. No. 10,960,349, issued Mar. 30, 2021, which is herein incorporated by reference in its entirety. Any entrained gases, including methanol, contained in the stripping gas exiting the stripper unit 104 may be fed to a second condenser 130 to condense any entrained methanol product of the stripping gas.

In an exemplary embodiment, column 101 may produce about 35%, such as from 25% to 40% of the high purity distillate methanol 114 produced by the chemical separations plant 100.

In some embodiments, column 101 can be a low-pressure column, e.g., operating at from 0 to about 0.5 bar (gauge pressure).

Column 105 is configured to receive at least a portion of the bottoms condensate fraction from column 101, e.g., through a side inlet as shown. Column 105 further comprises a reboiler 106 and a condensing unit 107. As shown, at least a portion of the bottoms output stream from column 105 is fed to reboiler 106. Reboiler 106 further comprises a heat exchanger that is coupled to and receives heat from an overhead vaporous fraction produced from column 108, wherein heat (e.g., heat of condensation from the overhead fraction 108a) is transferred to reboiled stream 106a in reboiler 106, to reboil the recycled portion of the bottoms condensate fraction received from column 105. Reboiler 106 returns heated stream 106a to column 105, producing a vaporous fraction and a bottoms condensate fraction inside column 105. The vaporous fraction from top of column 105 is collected by the condensing unit 107, condensed into high purity distillate methanol 114, and sent to storage or other desired use, while the bottoms condensate fraction settles to the bottom of column 105 where it is divided, with a portion recycled through reboiler 106 and another portion fed to column 108 (pre-heated in heat exchanger 119 with hot wastewater).

In some embodiments, column 105 may produce about 40%, such as from 30% to 50% of the high purity distillate methanol 114 produced by the chemical separations plant 100.

In some embodiments, column 105 can be a low-pressure column, e.g., operating at from 0 to about 0.5 bar (gauge pressure).

Column 108 is configured to receive at least a portion of the bottoms condensate fraction from column 105 through a side inlet. Column 108 further comprises a reboiler 109 and a condensing unit, e.g., associated with or integrated into reboiler 106. At least a portion of the bottom fraction from column 108 is fed to reboiler 109, which is configured to receive high-grade heat (e.g., at about 260° C. to about 280° C.) from an external heat source (e.g., hot gas from a methanol synthesis reactor of an ETL plant) through stream 110. The high-grade heat delivered to the reboiler 109 boils up the bottoms condensate fraction received from column 108. Reboiler 109 returns the heated condensate fraction to column 108, producing a vaporous fraction and a bottoms fraction (largely water,) inside third column 108. The vaporous fraction is used to provide heat to the reboiler 106 via a heat exchanger, wherein the heat of condensation is transferred to the reboiler 106 and the condensed high purity distillate methanol 114 is sent to storage or other desired use. The water bottoms fraction can be expelled from the plant as wastewater stream 121, prior to recovering heat from the stream in heat exchangers 119 and 120. The purpose of heat exchangers 119 and 120 is to recover heat from the wastewater from column 108. Hot wastewater is first employed in heat exchanger 119 to pre-heat the feed to column 108. The partially cooled wastewater is then utilized in a heat exchanger to pre-heat the crude methanol feed stream to column 101. Preheating the feed streams to columns 108 and 101, respectively, reduces the required heat duty for the separations plant, i.e. this heat integration minimizes the heat input requirement and helps reach zero or near zero duty distillation.

In some embodiments, high-grade heat stream 110 provides sufficient energy to boil off all or substantially all of the methanol remaining in the bottoms condensate fraction in bottoms stream 108b from column 108, so that substantially all methanol is recovered in stream 114. In some embodiments, column 108 may produce about 25%, such as from 20% to 30% of the high purity distillate methanol 114 produced by the chemical separations plant 100.

In some embodiments, column 108 can be a medium pressure column, operating at a higher pressure than columns 101 and 105. By way of example, third column 108 may operate at a pressure of about 3 to about 3.5 bar (gauge pressure).

In some embodiments, stream 110 comprises hot gas or hot fluids produced by an exothermic reaction, wherein stream 110 comprises high-grade heat. By way of example, stream 110 is cooled upon passage through reboiler 109 to produce stream 111, wherein stream 111 may comprise medium to low-grade heat. This feature of using high-grade heat to power the reboiler of the final column can be used in any of the embodiments described herein. Some embodiments can utilize direct process heating, wherein streams 110 and/or 111 are fed directly to a given reboiler or heat exchanger. The use of direct process heating, as opposed to heating medium loops can decrease the installation cost of a given system by eliminating the need to install a dedicated heating medium loop. Furthermore, the use of direct process heating can decrease the operating cost of a given system because direct process heating may reduce or eliminate the need for various pumps. Nevertheless, embodiments that do include a closed heating medium loop are possible, and examples of such are described herein.

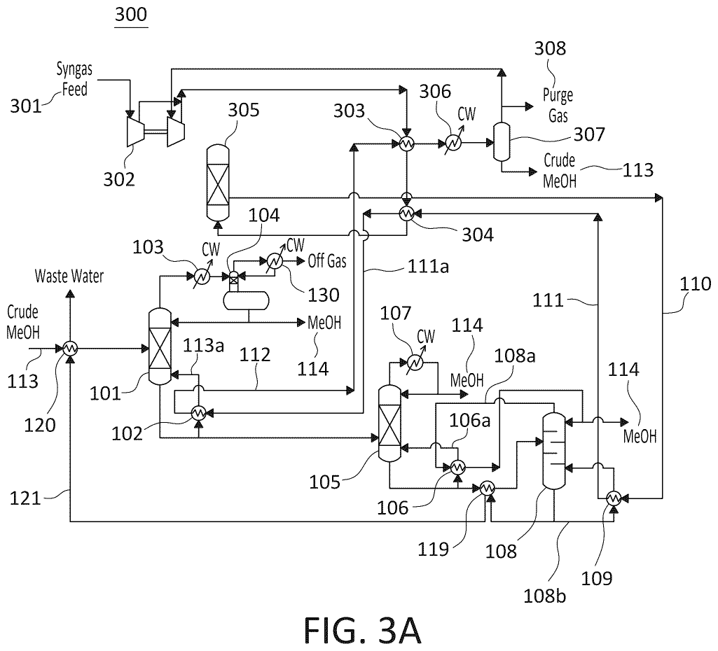

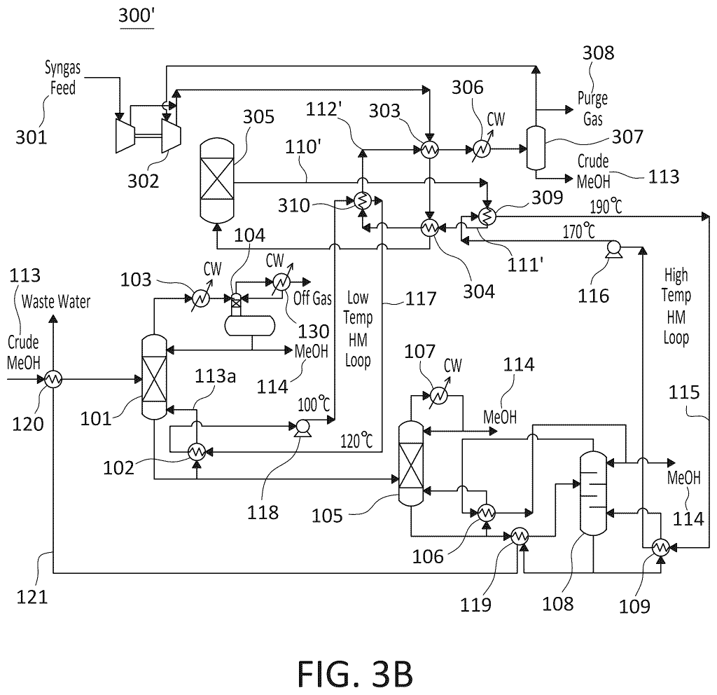

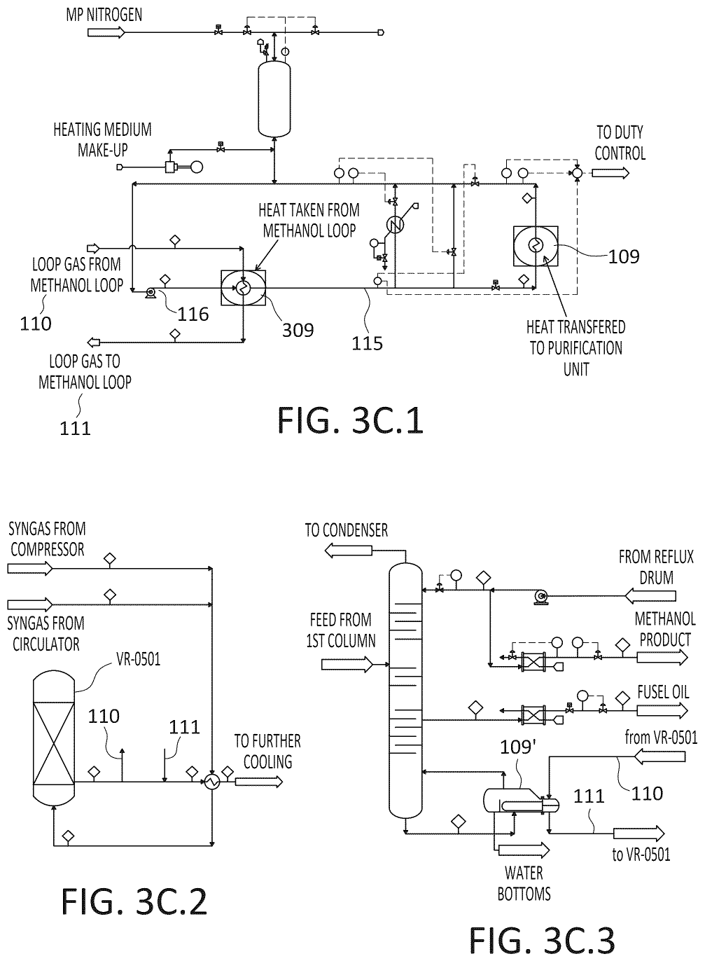

FIG. 3A illustrates a system for heat integration 300 similar to that shown in FIG. 1A, but which specifically shows integration with a chemical synthesis reactor (e.g., an ETL plant). Such a system may be described as a zero steam distillation 3-column purification system, with direct heating. Regarding chemical synthesis portion of the plant, a syngas feed 301 (e.g., H₂ and CO₂) may be fed into a compressor 302 and then heated up in the heat exchangers 303 and 304 The purpose of heat exchangers 303 and 304 is to preheat the syngas feed to the reactor 305, increasing the energy efficiency of the process. The heat exchangers sequence (downstream the reactor) 109-304-102-303 is arranged in such a way so as to maximize heat recovery from the ETL plant to the methanol separations plant. The syngas fee is finally provided to reactor 305 (e.g., a methanol conversion reactor), wherein an exothermic reaction takes place, converting syngas feed 301 into crude methanol resulting in stream 110, which comprises high-grade heat. Stream 110 is fed to a heat exchanger associated with reboiler 109, wherein high-grade heat is transferred to reboiler 109, resulting in cooled stream 111 that comprises medium to low-grade heat. Stream 111 is sent through heat exchanger 304 (e.g., to pre-heat the syngas stream before reaching reactor 305), and then as stream 111a to a heat exchanger associated with reboiler 102, wherein low-grade heat is transferred to reboiler 102, resulting in cooled stream 112. Stream 112 is sent to heat exchanger 303 (to pre-heat the syngas stream before heat exchanger 304) and then to condenser 306. The stream leaving condenser 306 enters a catch pot 307 where the top fraction 308 is purged from the plant and the bottom fraction (e.g., crude methanol) is fed to the chemical separations portion of the plant as process stream 113. By way of example the composition of crude methanol stream 113 may comprise 60-70% methanol, 30-40% water (by weight) plus a minor (e.g., negligible) amount of reaction byproducts.

It will be appreciated that the reboiler 102 comprises a low temperature window in exemplary chemical separations plant 100. Reboiler 102 receives the process stream with the highest methanol concentration and accordingly the lowest boiling point, thereby requiring the lowest temperature energy input to separate a significant portion of methanol from process stream 113. Therefore, stream 111a, which can provide sufficient heat to reboiler 102 (but not to reboiler 109), is coupled to reboiler 102. By way of example, in an embodiment, a minimum temperature required for reboiler 102 is 100° C., while reboiler 109 requires a minimum temperature of 160° C. In contrast to reboiler 102, the reboiler 109 (coupled to third column 108) receives a bottoms condensate fraction with the lowest overall concentration of methanol and accordingly the highest boiling point, requiring the highest temperature input to separate the last remaining methanol from the process stream 108b. Therefore, high-grade heat stream 110 is coupled to reboiler 109 in order to maximize the utility of the high-grade heat present in stream 110. By providing 3 columns for the separation (e.g., rather than 2), and separating steps that require less heat input from other steps that require more heat input, a low-grade heat, typically seen as waste heat, finds utility in the exemplary chemical separations plants described herein.

FIG. 1B shows an alternative operational scheme for chemical separations plant 100′, wherein chemical separations plant 100′ further comprises closed heating medium loops 115 and 117. As in FIG. 1A, chemical separations plant 100′ is designed to separate process stream 113 through a first column 101, a second column 105, and a third column 108. Column 101 is configured to receive process stream 113 through a side inlet. Column 101 further comprises a reboiler 102 and an associated condensing unit 103. At least a portion of the bottoms of column 101 is fed to reboiler 102, which is configured to receive low-grade heat from a heating medium loop 117. Heating medium loop 117 utilizes a plurality of heat exchangers to transfer heat from a low-grade heat source (e.g., low-grade heat from an ETL plant) to reboiler 102. Heating medium loop 117 further comprises a pump 118 and a heating medium, which can be any substance capable of transferring heat between heat exchangers, such as oil, water (e.g., demineralized water), or any other heating medium fluid. The low-grade heat delivered to reboiler 102 boils up process stream 113a received from column 101. Reboiler 102 returns heated process stream 113a to column 101, producing a vaporous fraction and a bottoms condensate fraction within column 101. The vaporous fraction is collected by the condensing unit 103 and a bottoms condensate fraction settles to the bottom of column 101, wherein the bottoms condensate fraction is either fed to column 105 or recycled through reboiler 102. As described in conjunction with FIG. 1A, the condensing unit 103 can be coupled to a stripper unit 104 that functions to remove dissolved gases from the condensed vaporous fraction, producing high purity distillate methanol 114 which is sent to storage or other desired use. Any entrained gases, including methanol, contained in the stripping gas exiting the stripper unit 104 may be fed to a second condenser 130 to condense any entrained methanol product of the stripping gas.

As described in conjunction with FIG. 1A, column 101 can be a low-pressure column. Column 101 can produce about 35% of the high purity distillate methanol 114 produced by the chemical separations plant 100′.

The second 105 column is configured to receive at least a portion of the bottoms condensate fraction from column 101 through a side inlet. As described in conjunction with FIG. 1A, column 105 further comprises a reboiler 106 and includes an associated condensing unit 107. At least a portion of the bottom fraction from column 105 is fed to reboiler 106. Reboiler 106 further comprises a heat exchanger that is coupled to a top fraction received from column 108, wherein the heat of condensation from the top fraction is transferred to reboiler 106 and used to boil up the bottoms condensate fraction received from column 105. Reboiler 106 returns the heated condensate fraction to column 105, producing a vaporous fraction and a bottoms condensate fraction inside column 105. The vaporous fraction from top of column 105 is collected by condensing unit 107, condensed into high purity distillate methanol 114, and sent to storage or other desired use. The bottoms condensate fraction settles at the bottom of column 105, wherein the bottoms condensate fraction is recycled through reboiler 106 and fed to column 108 (e.g., pre-heated in heat exchanger 119 with hot wastewater).

In some embodiments, column 105 produces about 40% of the high purity distillate methanol 114 produced by the chemical separations plant 100′. In some embodiments, column 105 can be a low-pressure column.

Similar to as described in conjunction with FIG. 1A, column 108 is configured to receive the bottoms condensate fraction from column 105 through a side inlet. Column 108 further comprises a reboiler 109. At least a portion of bottom outlet in column 108 is fed to reboiler 109, which is configured to receive high-grade heat from a heating medium loop 115. Heating medium loop 115 utilizes a plurality of heat exchangers to transfer heat from a high-grade heat source (e.g., a methanol conversion reactor of an ETL plant) to reboiler 109. Heating medium loop 115 may further include a pump and a heating medium, which can be any substance capable of transferring heat between heat exchangers, such as oil, water, another fluid. The high-grade heat delivered to the reboiler 109 boils up the bottoms condensate fraction received from column 108. Reboiler 109 returns the heated condensate fraction to column 108, producing a vaporous fraction and a bottoms fraction (largely water) inside column 108. The vaporous top fraction is conveyed to the reboiler 106 via a heat exchanger, wherein the heat of condensation is transferred to the reboiler 106 and the top fraction is eventually condensed into high purity distillate methanol 114, which sent to storage or other desired use. The bottoms fraction settles at the bottom of column 108, wherein the bottoms condensate fraction is either recycled through reboiler 109 or expelled from the plant as wastewater stream 121, prior to recovering heat from the stream in heat exchangers 119 and 120.

In some embodiments, heating medium loop 115 provides sufficient energy to boil off all or substantially all of the methanol remaining in the bottoms condensate fraction received from column 105, so that the resulting wastewater stream 121 is substantially free of methanol.

In some embodiments, column 108 can be a medium pressure column, and may produce about 25% of the high purity distillate methanol 114 produced by the chemical separations plant 100′.

FIG. 3B illustrates a system for heat integration 300′ similar to that shown in FIG. 1B, but which specifically shows integration with a chemical synthesis reactor (e.g., an ETL plant). Such a system may be described as a zero steam distillation 3-column purification system, with 2 heating medium loops that operate at different temperatures. Regarding the chemical synthesis portion of the plant, a syngas feed 301 (e.g., H₂ and CO₂) may be fed into a compressor 302 and then heat exchangers 303, 304 and then to reactor 305 (e.g., a methanol conversion reactor), wherein an exothermic reaction takes place, converting syngas feed 301 into crude methanol resulting in stream 110′, which comprises high-grade heat. Stream 110′ is fed to heat exchanger 309, wherein heat exchanger 309 is configured to deliver high-grade heat to heating medium loop 115, resulting in cooled stream 111′. Stream 111′ is sent through heat exchanger 304 (e.g., final preheat before reactor 305), and then to heat exchanger 310, wherein heat exchanger 310 is configured to transfer low-grade heat to heating medium loop 117, resulting in cooled stream 112′ Stream 112′ is sent to heat exchanger 303 (e.g., initial preheat before reactor 305) and then to condenser 306. The stream leaving condenser 306 enters a catch pot 307 where the top fraction 308 is purged and the bottom fraction is fed to the chemical separations plant as process stream 113 (e.g., crude methanol).

Heating medium loop 115 utilizes pump 116 to pump the heating medium from heat exchanger 309 to reboiler 109 and cooled heating medium from reboiler 109 to heat exchanger 309. Likewise, heating medium loop 117 utilizes pump 118 to pump heated heating medium from heat exchanger 310 to reboiler 102 and cooled heating medium from reboiler 102 to heat exchanger 310.

The use of one or more heating medium loops can simplify the design of each reboiler, simplify the heating control system of each reboiler, and lower the differential rating on the tube and shell side of the heat exchanger associated with the reboiler to prevent fouling.

FIGS. 3C.1, 3C.2, and 3C.3 schematically illustrate, within the normal heating medium configuration, the heat exchanger 309 and reboiler 109, and other associated equipment involved to direct transfer heat from the methanol loop or other chemical synthesis to the purification portion of the system. While a heating medium system can be used (where the standard scheme of FIG. 3C.1 is considered), where usually the heat from the methanol loop is transferred to purification via a heat carrier fluid (e.g., demineralized water) using the heat exchanger 309 and reboiler 109, alternatives are possible, where heat can be more directly transferred. In an example schematically summarized in FIGS. 3C.2 and 3C.3, the inlet temperature of reboiler 109′ (on the methanol loop side) may be about 260° C. to about 280° C. (stream 110) and the outlet temperature from reboiler 109′, may be about 190° C. to about 210° C. (stream 111). The water bottoms temperature (in the distillation section) may be at its boiling point, e.g., about 150° C. at the operating pressure. In this case the latent heat necessary to vaporize the required fluid for the medium pressure column is provided within the medium pressure column reboiler(s). Such direct heat arrangement reduces the necessity of equipment resulting in lower capital expense, since the full heating medium system (with all associated exchangers, piping and pumps) is not necessary. The total heat required for the purification section may be provided by two reboilers. The first may utilize the heat coming from the methanol loop and the second may use steam (or electricity depending on the configuration). Such a heat recovery arrangement minimizes the consumption of external utilities (steam or electricity) in normal operation. Such configurations are described herein.

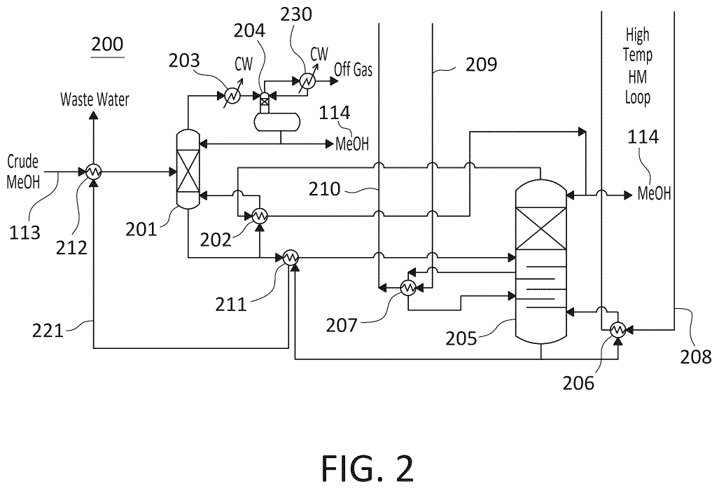

FIG. 2 illustrates an alternative configuration, that employs 2 separation columns, wherein the final column includes a side reboiler, rather than the 3 column configuration shown in FIGS. 1A-1B and FIGS. 3A-3B. FIG. 2 illustrates an exemplary operational scheme for such an exemplary chemical separations plant 200 separating a process stream 113 through a first column 201 and a second column 205. Column 201 is configured to receive process stream 113 (e.g., crude methanol) through a side inlet. Column 201 further comprises a reboiler 202 and an associated condensing unit 203. The reboiler 202 further comprises a heat exchanger that is coupled to a vaporous top fraction from column 205, wherein the heat of condensation from the top fraction from column 205 is transferred to reboiler 202 and used to boil up that portion of the bottom stream from column 201 that passes through reboiler 202, producing a vaporous top fraction and a bottoms condensate fraction inside column 201. The bottoms condensate fraction settles to the bottom of column 201, wherein the bottoms condensate fraction is either recycled through reboiler 202 or fed to column 205 (preheated in heat exchanger 211 with hot wastewater). The top fraction is collected by condensing unit 203. Condensing unit 203 is optionally coupled to a stripper unit 204 that functions to remove dissolved gases from the top fraction, producing high purity distillate methanol 114, which is sent to storage or other desired use. Any entrained gases, including methanol, contained in the stripping gas exiting the stripper unit 204 may be fed to a second condenser 230 to condense any entrained methanol product of the stripping gas.

In some embodiments, column 201 can be a low-pressure column.

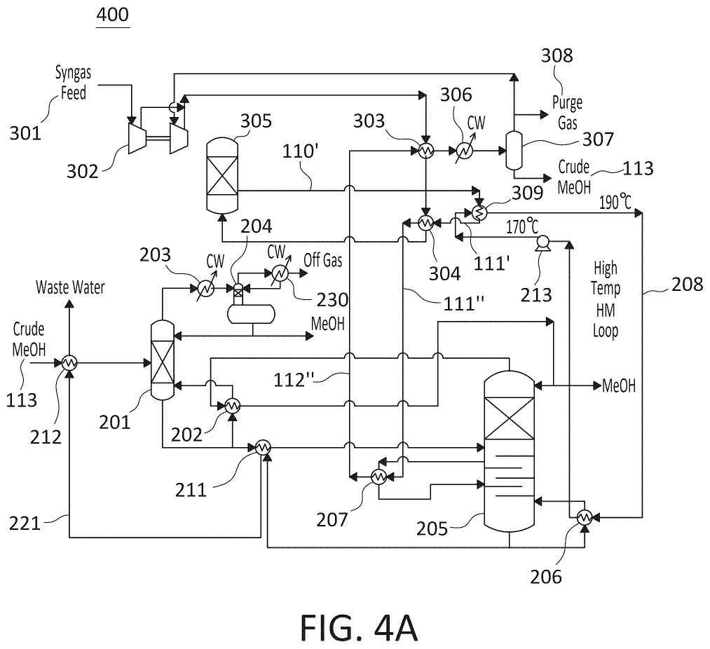

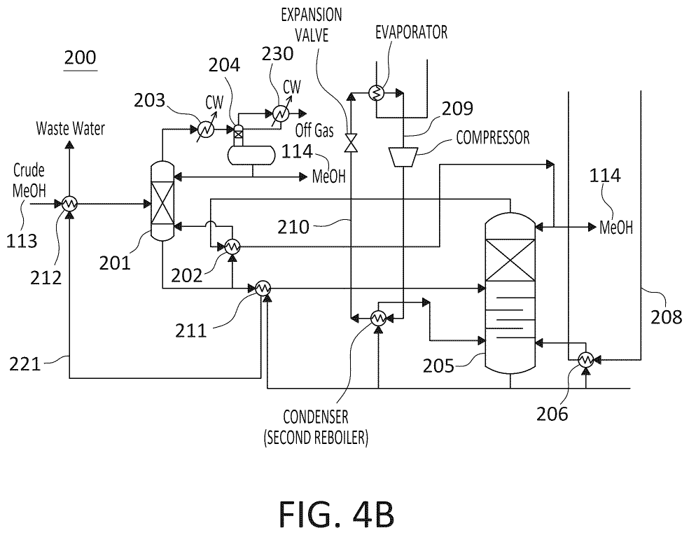

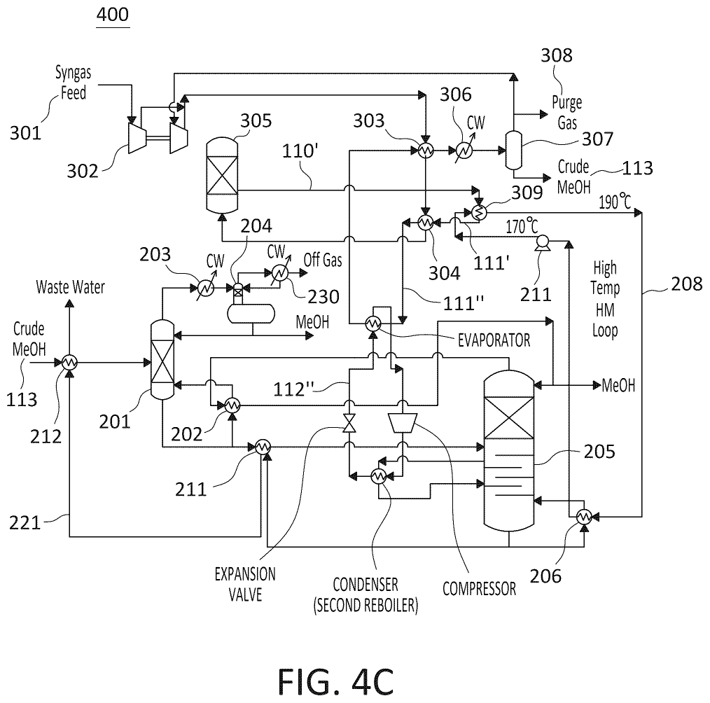

Column 205 is configured to receive the bottoms condensate fraction from column 201 through a side inlet. Column 205 further comprises a first reboiler 206, a second reboiler 207, and an associated condensing unit (e.g., in-line with reboiler 202). A bottom fraction from column 205 is fed to reboiler 206, which is configured to receive high-grade heat, e.g., from a heating medium loop 208. Heating medium loop 208 utilizes a plurality of heat exchangers to transfer heat from a high-grade heat source (e.g., a methanol conversion reactor of an ETL plant) to reboiler 206. Heating medium loop 208 further comprises a pump 211 (shown in FIG. 4A) and a heating medium, which can be any substance capable of transferring heat between heat exchangers, such as oil, water, or another fluid. While a closed heating medium loop is shown, it will be appreciated that a direct heating configuration can also be used, e.g., as shown and described in conjunction with FIGS. 1A and 3A. The high-grade heat delivered to reboiler 206 boils up the condensate fraction received from column 205. Reboiler 206 returns the heated condensate fraction to column 205, producing a vaporous top fraction and a bottoms fraction inside column 205. A side outlet in column 205 is fed to reboiler 207, which is configured to receive low-grade heat from an external heat source (e.g., waste heat from an ETL plant) through stream 209, boiling up a middle fraction moving up through column 205. Streams 209 and 210 may be either an open direct heating type scheme (e.g., as in FIG. 4A), or a closed heating loop. Reboiler 207 returns the heated middle fraction to a mid-portion of column 205 (hence the reboiler 207 is termed a “side reboiler), further contributing to the vaporous top fraction and the bottoms fraction inside column 205. The vaporous top fraction is sent to reboiler 202, wherein the heat of condensation is transferred to reboiler 202 and the top fraction is eventually condensed into high purity distillate methanol 114 and sent to storage or other desired use. The bottoms fraction settles to the bottom of column 205, wherein the bottoms fraction is either recycled through reboiler 206 or expelled from the plant as wastewater stream 221, prior to recovering heat from the stream in heat exchangers 211 and 212.

In some embodiments, column 205 can be a medium pressure column.

FIG. 4A illustrates a system for heat integration 400 similar to that shown in FIG. 2, but which specifically shows integration with a chemical synthesis reactor (e.g., an ETL plant). Such a system may be described as a zero steam distillation purification system, with a side reboiler. Regarding the chemical synthesis portion of the plant, a syngas feed 301 (e.g., H₂ and CO₂) may be fed into a compressor 302 and then via heat exchangers 303, 304 to methanol conversion reactor 305, wherein an exothermic reaction takes place, converting syngas feed 301 into crude methanol resulting in stream 110′ which comprises high-grade heat. Stream 110′ is fed to heat exchanger 309, wherein heat exchanger 309 is configured to deliver high-grade heat to heating medium loop 208, resulting in cooled stream 111′. Stream 111′ is sent through heat exchanger 304, and then to a heat exchanger associated with reboiler 207 to transfer low-grade heat to reboiler 207, resulting in cooled stream 112″. Stream 112″ is sent to heat exchanger 303 and then to condenser 306. The stream leaving condenser 306 enters a catch pot 307 where the top fraction 308 is purged and the bottom fraction is fed to the chemical separations plant as process stream 113 (e.g., crude methanol).

Heating medium loop 208 utilizes pump 213 to pump heated heating medium from heat exchanger 309 to reboiler 206 and cooled heating medium from reboiler 206 to heat exchanger 309. While FIG. 4A shows a direct heating configuration associated with delivery of low-grade heat to side reboiler 207, it will be appreciated a closed heating loop could alternatively be used (e.g., similar to loop 117 shown in FIG. 3B).

It may be appreciated that reboiler 207 (coupled to column 205) comprises a low temperature window in exemplary chemical separations plant 200. Reboiler 207 receives a liquid fraction that is drawn off the middle portion of column 205, wherein the liquid fraction is lower in temperature than the fraction at the bottom of column 205. In an embodiment, the temperature at the middle of the column (where a middle fraction is drawn off to side reboiler 207) is typically around 110° C. to 120° C., while the bottom of the column 205 is typically between 145° C. to 155° C. As a result, low-grade heat is sufficient to partially boil up this cooler fraction. Therefore stream 111″, which can provide sufficient heat to reboiler 207 but not to reboiler 206, is coupled to side reboiler 207.

The systems of heat integration as described herein improve the energy efficiency of the overall plant. Specifically, the effluent stream from reactor 305 is cooled by transferring high-grade heat and low-grade heat to strategically placed reboilers in a chemical separations plant, reducing the duty required to cool the product effluent stream from the reactor, while reducing the duty required to power the reboilers in the chemical separations portion of the plant. In some embodiments, the total heat required to perform a chemical separation process in chemical separations plants 100 and 200 is supplied by the integrated ETL or other exothermic chemical synthesis plant, by implementing systems such as those shown in FIGS. 3A, 3B and 4A. In these embodiments, the need for steam as a heat source is displaced, or completely eliminated. Further embodiments, such as those described below, describe alternative designs that can displace the need for steam as a heat source.

FIGS. 4B and 4C schematically illustrate systems similar to FIGS. 2 and 4A respectively, but in which a heat pump is incorporated to upgrade low-grade heat, without otherwise changing the heating medium loop of the system seen in FIGS. 2 and 4A. For example, such a system could be described as a 2-column (split-column) design incorporating a heat pump to upgrade low-grade heat. This scheme is based on FIG. 4A (a 2-column side-reboiler concept) where the previously mentioned side-reboiler is moved from the middle fraction to the bottoms fraction and is now a “second reboiler”, and instead of utilizing low-grade heat directly, the low-grade heat is upgraded through a heat pump circuit (e.g., comprising an evaporator, compressor, condenser, and an expansion valve) to approximately the same temperature level as the first reboiler. This adds a second reboiler that delivers a stream that is of similar quality (similar temperature) as the stream delivered by the first reboiler (e.g., at around 125° C. to 175° C., around 135° C. to 165° C. or around 145° C. to 155° C.). Due to the heat pump, a low-grade heat source can be used to contribute part of the heat needed in the distillation process, rather than requiring high-grade heat for the entire feed stream that would otherwise be fed into the column through the first reboiler. The use of the heat pump circuit can eliminate or reduce the use of import steam while making efficient use of the additional electricity needed to run the compressor of the heat pump circuit. FIG. 4B schematically illustrates such an exemplary chemical separations plant designed to utilize low-grade heat through implementation of a heat pump circuit, comprising an evaporator, a compressor, a condenser (second reboiler), and an expansion valve. FIG. 4C schematically illustrates a system that integrates the chemical separations plant of FIG. 4B with a methanol synthesis (e.g., ETL) plant. Such a system may be described as a zero steam distillation purification system, with a side reboiler.

Those of skill in the art will appreciate that additional heat pump schemes, as well as a variety of temperature ranges, refrigerants or refrigerant blends and the like can be used, etc. The use of embodiments incorporating use of a heat pump further the goal of providing a “fully electric plant” incorporating electrified heating in different embodiments. Compared to direct electricity-to-heat, a heat pump where electricity is fed to a compressor in the heat pump circuit has potential for significant operational expense savings due to thermodynamic upsides of moving around heat vs. creating it from electricity. Recent advances in high temperature heat pumps suitable for the temperature levels contemplated herein make such embodiments particularly attractive.

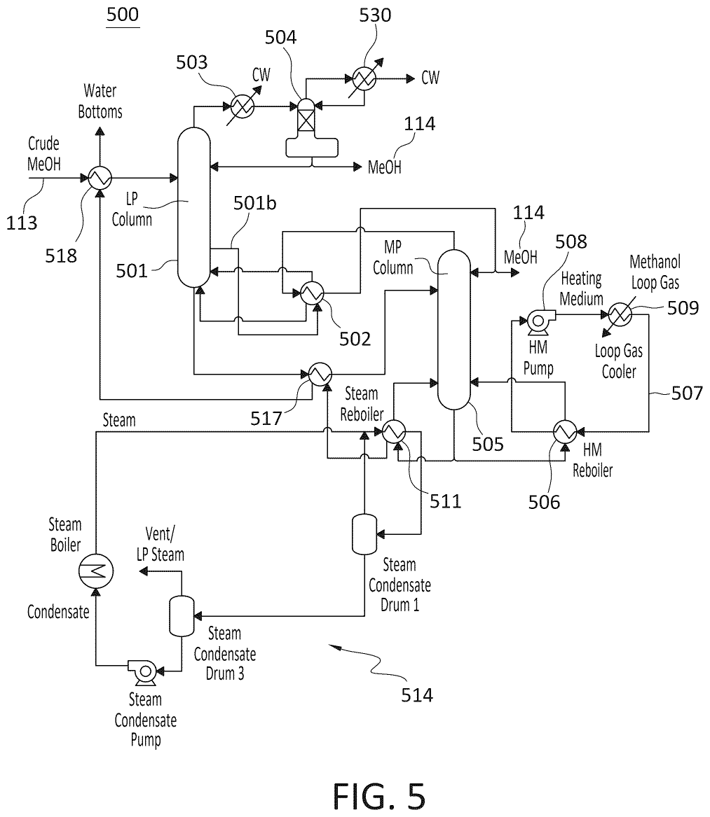

FIG. 5 illustrates an exemplary operational scheme for an exemplary chemical separations plant 500 separating a process stream 113 (e.g., crude methanol) through a first column 501 and a second column 505. Column 501 is configured to receive process stream 113 through a side inlet. Column 501 further comprises a reboiler 502 and an associated condensing unit 503. A bottom fraction from column 501 is fed to reboiler 502. Reboiler 502 includes a heat exchanger that provides heat to the material passing through reboiler 502, with the heat being provided by a vaporous top fraction from second column 505, wherein the heat of condensation from the vaporous top fraction from column 505 is transferred to reboiler 502 and used to boil up bottoms stream 501b, producing a vaporous fraction and a bottoms condensate fraction inside column 501. The vaporous top fraction is collected by condensing unit 503 and a bottoms condensate fraction settles to the bottom of column 501. A portion of the bottoms condensate fraction is fed to column 505 while another portion is recycled to column 501 through reboiler 502. The condensing unit 503 is optionally coupled to a stripper unit 504 that functions to remove dissolved gases from the condensed vaporous fraction, producing high purity distillate methanol 114, which is sent to storage or other desired use. Any entrained gases, including methanol, contained in the stripping gas exiting the stripper unit 504 may be fed to a second condenser 530 to condense any entrained methanol product of the stripping gas. In an embodiment, the stripping gas may comprise nitrogen, although it will be appreciated that others are also possible.

In some embodiments, column 501 can be a low-pressure column.

Column 505 is configured to receive the bottoms condensate fraction from column 501 through a side inlet. Column 505 further comprises a first reboiler (e.g., a heating medium reboiler) 506, a second reboiler (e.g., a steam reboiler) 511, and an associated condensing unit associated with or downstream from reboiler 502. A bottom fraction from column 505 is fed to reboiler 506, which is configured to receive high-grade heat from a heating medium loop 507. Heating medium loop 507 further comprises a pump 508 and a heating medium, which can be any substance capable of transferring heat between heat exchangers, such as oil, water, or another fluid, boiling up the condensate fraction received from column 505. Heating medium loop 507 utilizes heat exchanger 509 to transfer heat from a high-grade heat source (e.g., a methanol conversion reactor of an ETL plant) to the heating medium. Pump 508 circulates heated heating medium to reboiler 506 and cooled heating medium back to heat exchanger 509. The high-grade heat delivered to reboiler 506 boils up the condensate fraction received from column 505. Reboiler 506 returns the heated condensate fraction to column 505, producing a vaporous top fraction and a bottoms fraction inside column 505. Another portion of bottoms fraction from column 505 is fed to reboiler 511, which is configured to receive heat from steam heating infrastructure 514 (e.g., the illustrated steam boiler, steam condensate pump, and steam condensate drums), which is configured to boil and circulate and deliver heated steam to reboiler 511. The heat delivered to reboiler 511 boils up the condensate fraction received from column 505. Reboiler 511 returns the heated condensate fraction to column 505, further contributing to the vaporous top fraction and a bottoms fraction inside column 505. The vaporous top fraction is collected by a condensing unit associated with reboiler 502 (e.g., downstream therefrom), wherein the heat from such top fraction stream is transferred to reboiler 502 and the vaporous top fraction is condensed into high purity distillate methanol 114 and sent to storage or other desired use. The bottoms condensate fraction from column 505 is either expelled from the plant as wastewater or recycled through reboiler 506 and/or reboiler 511. The withdrawal of liquid could happen from the bottom of column 505 or from heat exchanger 506 as well, although perhaps not as convenient.

The configuration shown in FIG. 5 shows liquid sent separately to reboilers 506 and 511 (e.g., they are in hydrostatic balance, so the liquid can flow back and forth between 505, 506, and 511). In an embodiment, only the vapor from the reboilers is sent back to column 505. Liquid is withdrawn from reboiler 511 and expelled as wastewater after going through heat recovery in heat exchangers 517 and 518. Stated another way, reboiler 506 boils the bottoms from column 505. The vapor generated is sent to column 505. The remaining liquid is sent to reboiler 511. The vapor generated in reboiler 511 is sent to column 505. The remaining liquid that is not boiled in reboiler 511 is expelled as wastewater (after going through heat recovery in heat exchangers 517 & 518). It would alternatively be possible to send both the water and the vapor from reboiler 506 back to column 505, and then the liquid is sent separately to reboiler 511. Liquid and vapor from reboiler 511 is sent to column 505 and then the liquid is finally withdrawn from the bottom of column 505, although this may not provide any additional benefit. Other configurations where the water is withdrawn from reboiler 506 and expelled as waste water would also of course also be possible.

An exemplary purpose of illustrated heat exchangers 517 and 518 is to recover heat from the water bottoms of column 505. For example, the configuration may be that the hot water bottoms from 505 are sent to heat exchanger 517, where heat is transferred from hot water bottoms from 505 to the colder bottom condensate fraction from column 501. The hot bottom condensate fraction from heat exchanger 517 is then sent as feed to column 505.

Heat exchanger 518 receives partially cooled water bottoms from heat exchanger 517 (originating from column 505) and the heat is transferred to the cold crude methanol in stream 113. Hot stream 113 is then sent as feed to column 501. This heat recovery reduces the amount of heat that is required to be supplied to column 505 through heat exchangers 511 and 506.

Chemical separations plant 500 is an exemplary chemical separations plant that relies on steam related infrastructure as a heat source. However, a reliable source of steam may be difficult to attain and/or may cut into carbon emissions savings. Additional embodiments described below displace the need for steam related infrastructure and allow for the integration of electrical sources of energy, including renewable sources of energy.

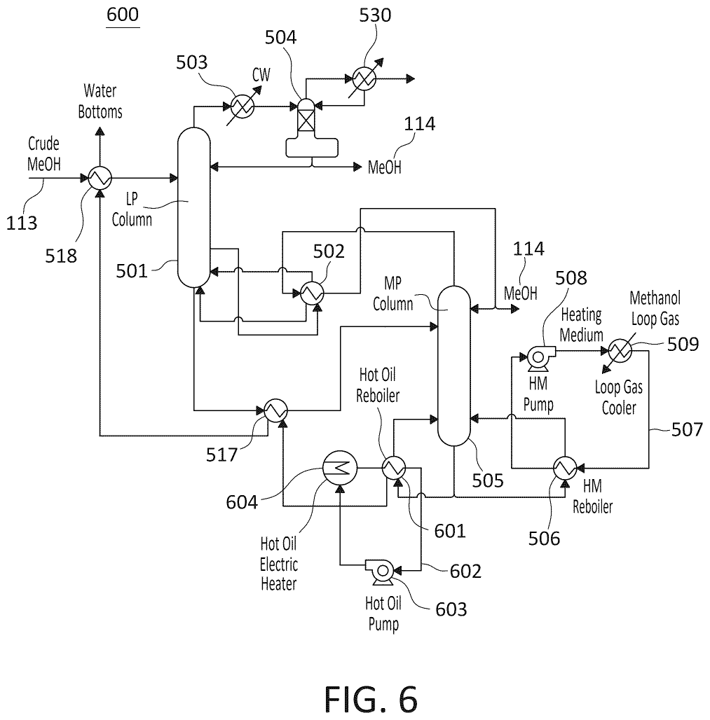

FIG. 6 illustrates an exemplary operational scheme for an exemplary chemical separations plant 600 that is an electrified alternative to chemical separations plant 500. As illustrated in FIG. 6, chemical separations plant 600 replaces reboiler 511 and the related steam heating infrastructure 514 with reboiler 601, wherein reboiler 601 is configured to receive heat from a hot oil loop 602. Hot oil loop 602 further comprises pump 603 and electric heater 604, wherein heater 604 heats oil in hot oil loop 602 and pump 603 circulates heated oil from heater 604 to reboiler 601 and cooled oil from reboiler 601 to heater 604. The heat delivered to reboiler 601 boils up the condensate fraction received from column 505. Reboiler 601 returns the heated condensate fraction to column 505, further contributing to the vaporous top fraction and a bottoms fraction inside column 505. In an embodiment only vapor from 601 is sent to column 505. Liquid is withdrawn from reboiler 601 and expelled as wastewater after heat recovery in heat exchangers 517 and/or 518. The vaporous top fraction is sent to reboiler 502, wherein the heat available from such stream is transferred to reboiler 502 and the top fraction is condensed into high purity distillate methanol 114 and sent to storage or other desired use. The bottoms condensate fraction from column 505 is either expelled from the plant as wastewater or recycled through reboiler 506 and/or reboiler 601. FIG. 6 may show only liquid being withdrawn from reboiler 601, although liquid could also be withdrawn from column 505 or reboiler 506, if desired.

In some embodiments, heater 604 runs on electricity produced by renewable sources, reducing any associated carbon emissions footprint.

Chemical separations plant 600 displaces the need for steam by replacing heating infrastructure that relies on steam as a heat source with a hot oil loop powered by an electric heater. Furthermore, because heating elements of electric heater 604 do not come in direct contact with the contents of reboiler 601, plant 600 minimizes possible thermal degradation of the process stream 113.

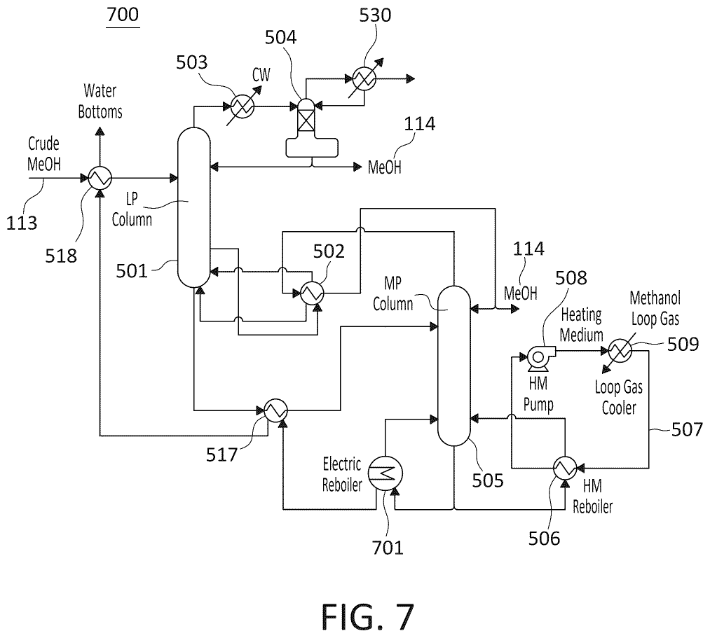

FIG. 7 illustrates an exemplary operational scheme for an exemplary chemical separations plant 700 that is an electrified alternative to chemical separations plant 500. As illustrated in FIG. 7, chemical separations plant 700 replaces reboiler 511 and the related steam heating infrastructure 514 with reboiler 701, wherein reboiler 701 further comprises an internal electric heater, which boils up the condensate fraction received from column 505. Reboiler 701 returns the heated condensate fraction to column 505, further contributing to the vaporous top fraction and a bottoms fraction inside column 505. The top fraction and bottom fraction are otherwise treated in the same way as already described in conjunction with FIGS. 5-6.

Chemical separations plant 700 displaces the need for steam by replacing heating infrastructure that relies on steam as a heat source with a reboiler that comprises an internal electric heater. Because of its simple design with limited infrastructure, chemical separations plant 700 can greatly reduce installation and maintenance costs.

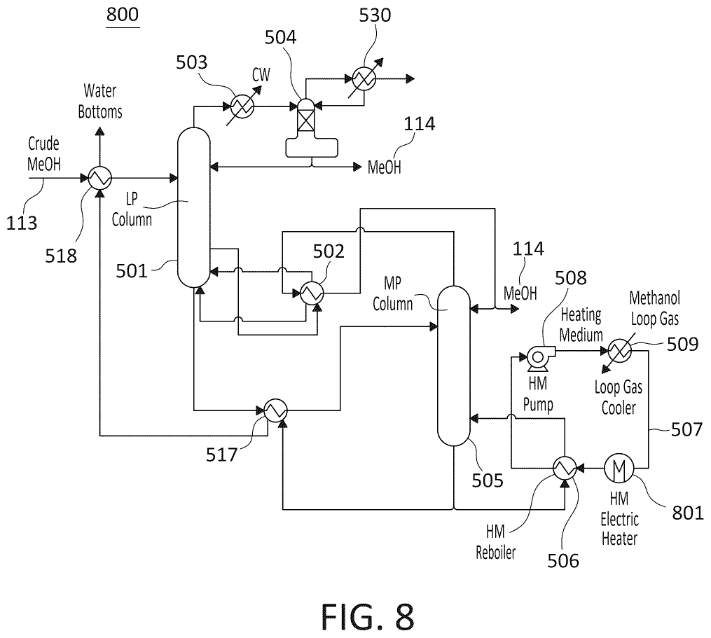

FIG. 8 illustrates an exemplary operational scheme for an exemplary chemical separations plant 800 that is an electrified alternative to chemical separations plant 500. As illustrated in FIG. 8, chemical separations plant 800 eliminates reboiler 511 and the related steam heating infrastructure 514 and adds electric heater 801 to heating medium loop 507, wherein electric heater 801 provides additional heat to the heating medium loop and to reboiler 506.

Chemical separations plant 800 displaces the need for steam by replacing heating infrastructure that relies on steam as a heat source with an additional electric heater that is installed in the heating medium loop. This allows a source of electrical energy to contribute to a chemical separation process in chemical separations plant 800.

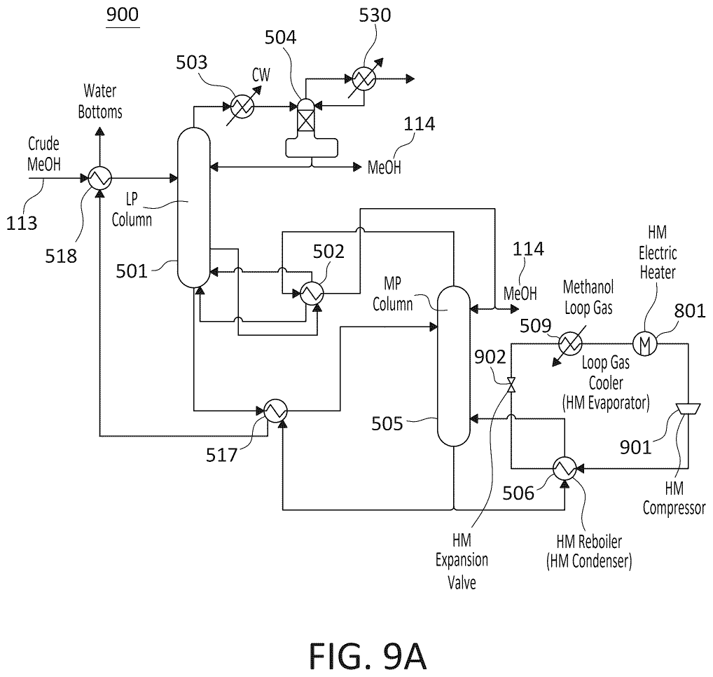

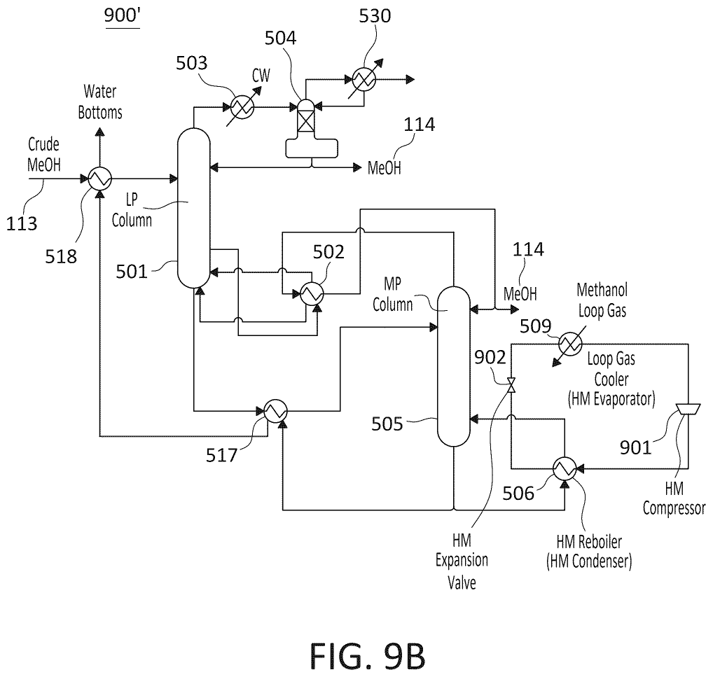

FIGS. 9A-9B illustrate additional exemplary operational schemes for exemplary chemical separations plants 900 and 900′ that are electrified alternatives to chemical separations plant 500. As illustrated in FIGS. 9A-9B, chemical separations plant 900/900′ eliminates reboiler 511 and the related steam heating infrastructure 514 and adds (e.g., electric) compressor 901 to heating medium loop 507 (which now functions as a heat pump), wherein compressor 901 increases the pressure of the heating medium. The heat from loop gas cooler 509, utilized to evaporate the heating medium, can optionally be supplemented with heat from an electric heater 801 (FIG. 9A) before the evaporated heating medium is upgraded to a higher temperature in compressor 901, allowing it to be utilized in reboiler 506. FIG. 9B shows a system 900′ that does not include the electric heater 801, while FIG. 9A shows a system 900 that does include such an optional electric heater 801. Any fluid capable of efficiently absorbing and releasing heat as they undergo repeated phase changes (from liquid to vapor and vice versa) may be utilized as a heating medium fluid in the heating medium loop. The configuration shown in FIG. 9B without such an electric heater 801 may be more practical, and simpler.

Chemical separations plant 900/900′ displaces the need for steam by replacing heating infrastructure that relies on steam as a heat source by adding (e.g., electric) compressor 901 and expansion valve 902, which allows the heating medium loop to function as a heat pump. This allows a source of electrical energy to contribute to a chemical separation process in chemical separations plant 900/900′.

Features of the disclosed embodiments may be combined or arranged for achieving particular advantages as would be understood from the disclosure by one of ordinary skill in the art. Similarly, features of the disclosed embodiments may provide independent benefits applicable to other examples not detailed herein.

It is to be understood that not necessarily all objects or advantages may be achieved under any embodiment of the disclosure. Those skilled in the art will recognize that system and method may be embodied or carried out in a manner that achieves or optimizes one advantage or group of advantages as taught without achieving other objects or advantages as taught or suggested.

The skilled artisan will recognize the interchangeability of various disclosed features. Besides the variations described, other known equivalents for each feature can be mixed and matched by one of ordinary skill in this art to make or use a heat, steam and/or other heated working fluid integration under principles of the present disclosure. It will be understood by the skilled artisan that the features described may be adapted to other systems and processes. Hence this disclosure and the embodiments and variations thereof are not limited to methanol synthesis processes or to specific partner plants but can be utilized by integrating heat between any exothermic chemical process that generates waste heat with any partner process that generates high value steam or another high value working fluid/heating medium.

Although this disclosure describes certain exemplary embodiments of chemical separations plants, and examples of heat integration with partner plants, it therefore will be understood by those skilled in the art that the present disclosure extends beyond the specifically disclosed embodiments to other alternative embodiments and/or uses of the disclosure and obvious modifications and equivalents thereof. It is intended that the present disclosure should not be limited by the particular disclosed embodiments described above.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. As used herein, the term “between” includes any referenced endpoints. For example, “between 2 and 10” includes both 2 and 10.

1. A chemical separations plant comprising:
a first column, a second column, and a third column, wherein:
the first column is configured to receive a chemical process stream, the first column further comprising at least one associated condensing unit, for example, located at an overhead portion of the first column, and at least one reboiler configured to receive low-grade heat from a low-grade heat source;
the second column is configured to receive at least a portion of a bottoms condensate fraction from the first column, the second column further comprising at least one associated condensing unit, for example, located at an overhead portion of the second column, at least one reboiler configured to receive heat from a top fraction of the third column; and
the third column is configured to receive at least a portion of a bottoms condensate fraction from the second column, the third column further comprising at least one associated condensing unit, for example, located at an overhead portion of the third column, and at least one reboiler configured to receive high-grade heat from a high-grade heat source.

2. The chemical separations plant of claim 1, wherein the first column further comprises at least one stripper unit located at the overhead portion of the first column.

3. The chemical separations plant of claim 1, wherein the chemical process stream comprises crude methanol.

4. The chemical separations plant of claim 3, wherein the first column produces from about 25% to about 40% of purified methanol produced by the chemical separations plant.

5. The chemical separations plant of claim 3, wherein the second column produces from about 30% to about 50% of purified methanol produced by the chemical separations plant.

6. The chemical separations plant of claim 3, wherein the third column produces from about 20% to about 30% of purified methanol produced by the chemical separations plant.

7. The chemical separations plant of claim 1, wherein the first column is a low-pressure column.

8. The chemical separations plant of claim 1, wherein the second column is a low-pressure column.

9. The chemical separations plant of claim 1, wherein the third column is a medium pressure column.

10. The chemical separations plant of claim 1, wherein the high-grade heat source is a stream of hot gas or other hot fluid produced by a chemical synthesis plant.

11. The chemical separations plant of claim 1, wherein the low-grade heat source is a stream of hot gas or other hot fluid produced by a chemical synthesis plant, wherein any high-grade heat initially present in the stream of hot gas or other hot fluid has been at least partially depleted.

12. The chemical separations plant of claim 10, wherein a heating medium loop transfers high-grade heat from the high-grade heat source to the at least one reboiler of the third column.

13. The chemical separations plant of claim 11, wherein a heating medium loop transfers low-grade heat from the low-grade heat source to the at least one reboiler of the first column.

14. The chemical separations plant of claim 11, wherein the low-grade heat source from which heat is recovered is a hot wastewater stream.

15. A system for heat integration of (i) a chemical synthesis plant which generates low-grade heat and high-grade heat from an exothermic reaction and (ii) the chemical separations plant of claim 1, the system comprising:
a reactor for carrying out the chemical synthesis configured to produce a process stream,
a first heat exchanger configured to transfer high-grade heat from the process stream to the reboiler of the third column, and
a second heat exchanger configured downstream from the first heat exchanger and configured to transfer remaining low-grade heat from the process stream to the reboiler of the first column.

16. A chemical separations plant comprising:
a first column configured to receive a chemical process stream, wherein the first column further comprises at least one condensing unit, for example, located at an overhead portion of the first column, and at least one reboiler;
a second column configured to receive at least a portion of a bottoms condensate fraction from the first column, wherein the second column further comprises at least one condensing unit, for example, located at an overhead portion of the second column, a first reboiler associated with a bottoms fraction of the second column, and a second reboiler associated with a middle fraction of the second column; and
a plurality of heat exchangers, wherein:
a first heat exchanger is configured to deliver a source of high-grade heat to the first reboiler of the second column,
a second heat exchanger is configured to deliver a source of low-grade heat to the second reboiler of the second column, and
a third heat exchanger is configured to deliver heat from a top fraction produced from the second column to the at least one reboiler of the first column.

17. The chemical separations plant of claim 16, wherein the first column further comprises at least one stripper unit located at the overhead portion of the first column.

18. The chemical separations plant of claim 16, wherein the chemical process stream comprises crude methanol.

19. The chemical separations plant of claim 16, wherein the first column is a low-pressure column.

20. The chemical separations plant of claim 16, wherein the second column is a medium pressure column.

21. A system for heat integration of (i) a chemical synthesis plant which generates low and high-grade heat from an exothermic reaction and (ii) the chemical separations plant of claim 16, the system comprising:
a reactor for carrying out the chemical synthesis configured to produce a process stream,
a first heat exchanger configured to transfer high-grade heat from the process stream to the first reboiler of the second column, and
a second heat exchanger configured downstream from the first heat exchanger and configured to transfer remaining low-grade heat from the process stream to the second reboiler of the second column.

22. A chemical separations plant comprising:
a first column configured to receive a chemical process stream, the first column further comprising, for example, at least one condensing unit located at an overhead portion of the first column, and at least one reboiler;
a second column configured to receive at least a portion of a bottoms condensate fraction from the first column, the second column further comprising at least one condensing unit, for example, located at an overhead portion of the second column, and at least one reboiler;
a heat exchanger configured to transfer heat to the at least one reboiler of the first column from a top fraction produced from the second column; and
an electric heat source configured to provide heat to the at least one reboiler of the second column.

23. The chemical separations plant of claim 22, wherein the electric heat source comprises a hot oil loop comprising tubing coupled to the at least one reboiler of the second column, a pump, and an electric heater.

24. The chemical separations plant of claim 22, wherein the electric heat source comprises an additional reboiler that further comprises an internal electric heater.

25. The chemical separations plant of claim 22, wherein the electric heat source comprises a heating medium loop comprising tubing coupled to the at least one reboiler of the second column, a pump, and an electric heater.

26. The chemical separations plant of claim 22, wherein the electric heat source comprises a heat pump comprising tubing coupled to the at least one reboiler of the second column, a compressor, and an expansion valve.

27. The chemical separations plant of claim 22, wherein the electric heat source does not comprise steam.

28. The chemical separations plant of claim 22, wherein the chemical separations plant is a methanol distillation plant.

29. The chemical separations plant of claim 22, wherein the first column further comprises at least one stripper unit located at an overhead portion of the first column.
